(19) United States
(12) Patent Application Publication
(10) Pub. No.: US 2024/0074798 A1
(43) Pub. Date: Mar. 7, 2024

(54) Repair Plate System

(71) Applicant: Tyber Medical LLC, Bethlehem, PA (US)

(72) Inventor: Robert Flavin, Enniskerry (IE)

(73) Assignee: Tyber Medical LLC, Bethlehem, PA (US)

(21) Appl. No.: 18/233,348

(22) Filed: Aug. 14, 2023

Related U.S. Application Data
(60) Provisional application No. 63/402,967, filed on Sep. 1, 2022.

(51) Int. Cl. A61B 17/72

(52) U.S. Cl. CPC A61B 17/725

(57) ABSTRACT

A repair plate system includes a body having a distal nail portion configured for implantation into a limb bone and a proximal plate portion attached to the distal nail portion. A first plurality of fixation screws are configured for insertion into the distal nail portion and a second plurality of fixation screws are configured for insertion into the proximal plate portion.

### BACKGROUND OF THE INVENTION

### Field of the Invention

The invention relates to a plate that is used to repair a broken or fractured limb proximate to a joint

### Description of the Related Art

The use of current plates and methods for repairing fractures of limbs, such as humeri and femurs, proximate to their ball joints, particularly in the elderly, often results in delayed or non-union of the bone pieces, depending on the fracture type and Calcar involvement. Also the inability to non weight bear among the elderly leads to loss of fixation.

It would be beneficial to provide a repair plate that can enhance the union and subsequent intergrowth of fractured bone portions.

### SUMMARY OF THE INVENTION

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

In one embodiment, the present invention is A repair plate system including a body having a distal nail portion configured for implantation into a limb bone and a proximal plate portion attached to the distal nail portion. A first plurality of fixation screws are configured for insertion into the distal nail portion and a second plurality of fixation screws are configured for insertion into the proximal plate portion.

In another embodiment, the present invention is a repair plate system comprising a body having a distal nail portion configured for implantation along a limb bone and a proximal plate portion attached to the distal nail portion. A plurality of fixation screws is configured for insertion into the proximal plate portion and a capture screw is configured to act as a gusset support for one of the plurality of fixation screws.

In still another embodiment, the present invention is a repair plate system comprising a body having a distal nail portion configured for implantation along a limb bone and a proximal plate portion attached to the distal nail portion. A first set of fixation screws is configured for insertion into the body and a second set of fixation screws configured for insertion into the body. A capture screw is configured to act as a gusset support for one of the sets of fixation screws.

### BRIEF DESCRIPTION OF THE DRAWINGS

The accompanying drawings, which are incorporated herein and constitute part of this specification, illustrate the presently preferred embodiments of the invention, and, together with the general description given above and the detailed description given below, serve to explain the features of the invention. In the drawings:

FIG. 1 is a front elevational view of an inside-out nail for femur repair according to an exemplary embodiment of the present invention;

FIG. 2 is a side elevational view of the nail of FIG. 1;

FIG. 3 is a rear elevational view of the nail of FIG. 1;

FIG. 4 is a top perspective view of the nail of FIG. 1;

FIG. 5 is a bottom perspective view of the nail of FIG. 1;

FIG. 6 is a perspective view of the nail of FIG. 1 implanted into a femur;

FIG. 7 is a side elevational view of the nail and femur of FIG. 6;

FIG. 8 is a front elevational view of the nail and femur of FIG. 6;

FIG. 9 is a rear elevational view of the nail and femur of FIG. 6;

FIG. 10 is a top perspective view of the nail and femur of FIG. 6;

FIG. 11 is a bottom perspective view of the nail and femur of FIG. 6;

FIG. 12 is a perspective view of an inside-out nail for humerus repair according to an exemplary embodiment of the present invention;

FIG. 13 is a front elevational view of the nail of FIG. 12;

FIG. 14 is a side elevational view of the nail of FIG. 12;

FIG. 15 is a rear elevational view of the nail of FIG. 12;

FIG. 16 is a top perspective view of the nail of FIG. 12;

FIG. 17 is a perspective view of the nail of FIG. 12 implanted into a humerus;

FIG. 18 is a side elevational view of the nail and humerus of FIG. 17;

FIG. 19 is a rear elevational view of the nail and humerus of FIG. 17;

FIG. 20 is a front elevational view of the nail and humerus of FIG. 17; and

FIG. 21 is a top perspective view of the nail and humerus of FIG. 17;

FIG. 22 is a side elevational view of an alternative embodiment of a nail according to the present invention implanted in a tibia;

FIG. 23 is a front elevational view of the nail and tibia of FIG. 1;

FIG. 24 is a perspective view of the nail and tibia of FIG. 22;

FIG. 25 is another perspective view of the nail and tibia of FIG. 21;

FIG. 26 is a perspective view of a screw blade capture system according to an alternative exemplary embodiment of the present invention;

FIG. 27 is a side elevational view of the system of FIG. 26;

FIG. 28 is a top perspective view of the system of FIG. 26;

FIG. 29 is a perspective view of the system of FIG. 26 used on a tibia;

FIG. 30 is a side elevational view of the system of FIG. 26 attached to the tibia of FIG. 29; and

FIG. 31 is a top perspective view of the system of FIG. 26 attached to the tibia of FIG. 29.

### DETAILED DESCRIPTION

U.S. Provisional Patent Application Ser. No. 63/402,961, entitled “Ligament/Tendon Suture with Paired Anchor System”, filed on this date and invented by this inventor (Attorney Docket No. TYB-056); U.S. Provisional Patent Application Ser. No. 63/402,962, entitled “Screw Capture System for Calcaneal Fracture”, filed on this date and invented by this inventor (Attorney Docket No. TYB-057); U.S. Provisional Patent Application Ser. No. 63/402,965, entitled “Screw Blade Capture System”, filed on this date and invented by this inventor (Attorney Docket No. TYB-058); U.S. Provisional Patent Application Ser. No. 63/402,964, entitled “Talar Repair Jig”, filed on this date and invented by this inventor (Attorney Docket No. TYB-060); U.S. Provisional Patent Application Ser. No. 63/402,969971, entitled “Syndesmosis Repair Jig Assembly and Method of Use”, filed on this date and invented by this inventor (Attorney Docket No. TYB-061); U.S. Provisional Patent Application Ser. No. 63/402, entitled “Distal Tibial Osteotomy System”, filed on this date and invented by this inventor (Attorney Docket No. TYB-062); U.S. Provisional Patent Application Ser. No. 63/402,963, entitled “External Fixation System”, filed on this date and invented by this inventor (Attorney Docket No. TYB-063); and U.S. Provisional Patent Application Ser. No. 63/402,970, entitled “Achilles Tendon Repair Jig”, filed on this date and invented by this inventor (Attorney Docket No. TYB-064), are all incorporated herein by reference in their entireties.

In the drawings, like numerals indicate like elements throughout. Certain terminology is used herein for convenience only and is not to be taken as a limitation on the present invention. The terminology includes the words specifically mentioned, derivatives thereof and words of similar import. The embodiments illustrated below are not intended to be exhaustive or to limit the invention to the precise form disclosed. These embodiments are chosen and described to best explain the principle of the invention and its application and practical use and to enable others skilled in the art to best utilize the invention.

Reference herein to “one embodiment” or “an embodiment” means that a particular feature, structure, or characteristic described in connection with the embodiment can be included in at least one embodiment of the invention. The appearances of the phrase “in one embodiment” in various places in the specification are not necessarily all referring to the same embodiment, nor are separate or alternative embodiments necessarily mutually exclusive of other embodiments. The same applies to the term “implementation.”

As used in this application, the word “exemplary” is used herein to mean serving as an example, instance, or illustration. Any aspect or design described herein as “exemplary” is not necessarily to be construed as preferred or advantageous over other aspects or designs. Rather, use of the word exemplary is intended to present concepts in a concrete fashion.

Additionally, the term “or” is intended to mean an inclusive “or” rather than an exclusive “or”. That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more” unless specified otherwise or clear from context to be directed to a singular form.

Unless explicitly stated otherwise, each numerical value and range should be interpreted as being approximate as if the word “about” or “approximately” preceded the value of the value or range.

The use of figure numbers and/or figure reference labels in the claims is intended to identify one or more possible embodiments of the claimed subject matter in order to facilitate the interpretation of the claims. Such use is not to be construed as necessarily limiting the scope of those claims to the embodiments shown in the corresponding figures.

The present invention is an inside-out femoral nail that can be used to repair osteoporotic fractures, to perform a proximal femoral osteotomy, and for treatment and repair of unstable subtrochanteric or Calcar extension fractures using a minimally invasive technique.

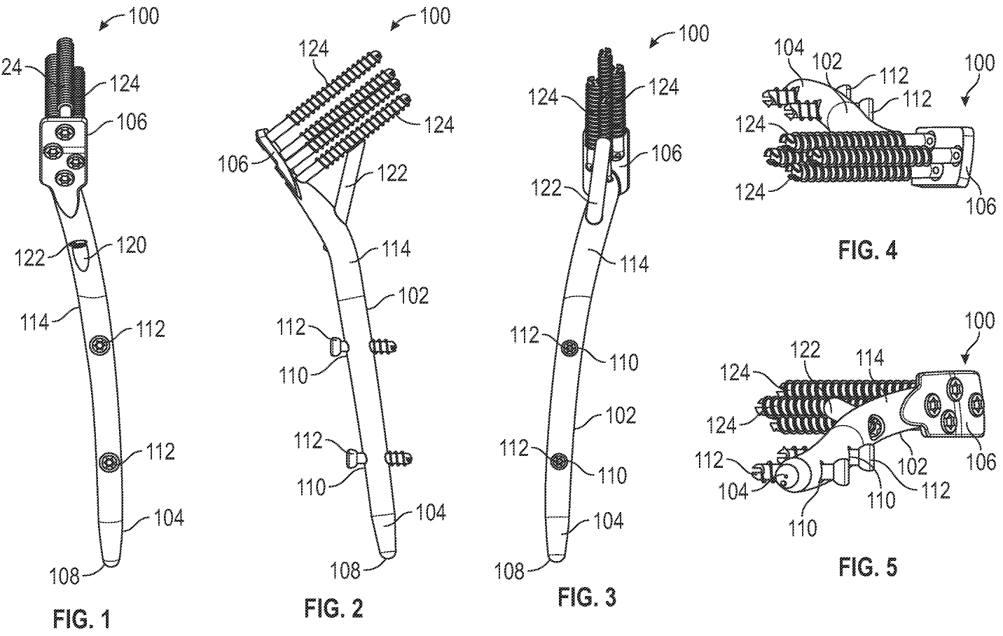

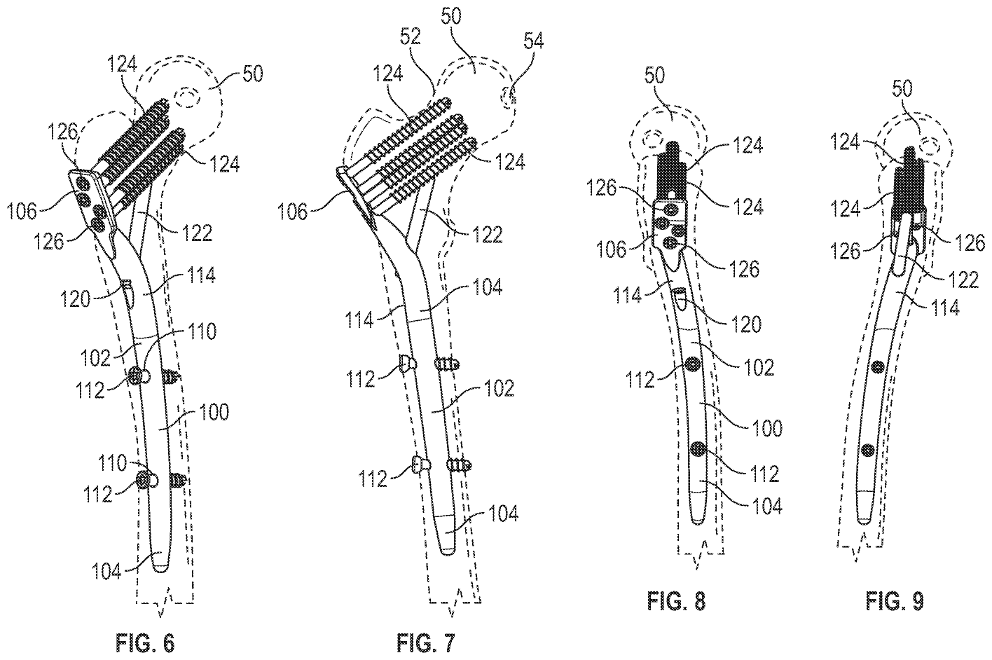

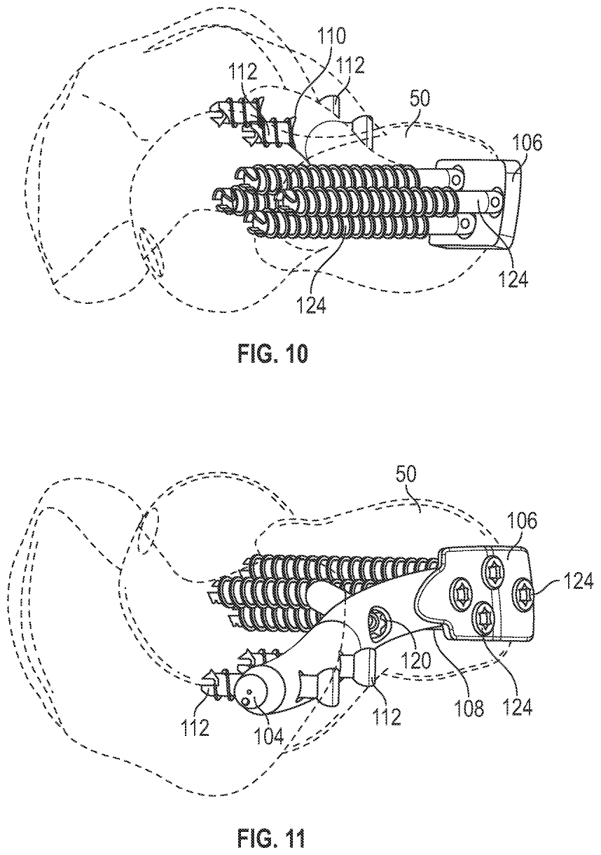

Referring to FIGS. 1-5, an inside-out femoral nail 100 (“nail 100”) is shown. Nail 100 is shown after implantation in a femur 50 in FIGS. 6-11. Nail 100 includes an elongate body 102 having a distal end 104 and a proximal end 106. Distal end 104 is implanted into femur 50, as shown in FIGS. 6-11, while proximal end 106 remains outside of femur 50.

Body 102 is generally cylindrical toward distal end 104, where distal end 104 terminates in a tapered tip 108. Distal end 104 includes a plurality of through-openings 110 that allow for the insertion of fixation screws 112 therethrough. Fixation screws 112 extend through femur 50, into through-openings 110, and back into femur 50 to secure distal end 104 to femur 50.

A mid-portion 114 between distal end 104 and proximal end 106 is curved to match the anatomy of femur 50 and includes a through-opening 120 for securing a capture screw 122. Capture screw 122 can be used to support calcar unstable structures, acting as a gusset support for a proximal fixation screw 124 to reduce any leverage provided by screw 124 due to its length.

Proximal end 106 is a generally flat plate having a plurality of through-openings 126 formed therethrough. Screws 124 are longer than screws 112 and are inserted through through-openings 126, through neck 52 and into head 54 of femur 50, as best seen in FIG. 7.

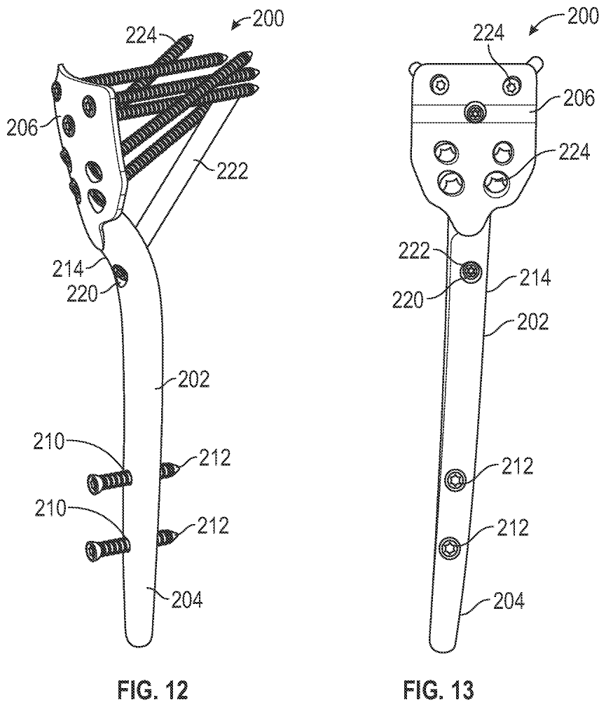

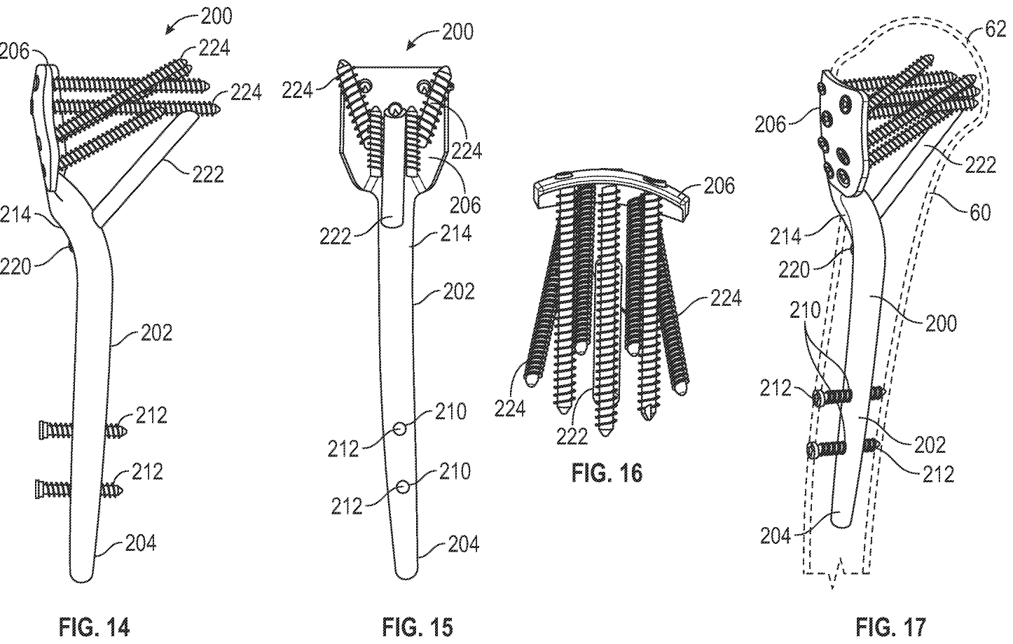

Referring to FIGS. 12-16, an inside-out humerus nail 200 (“nail 200”) is shown. Nail 200 is shown after implantation in a humerus 60 in FIGS. 17-21. Nail 200 is similar to nail 100 and includes an elongate body 202 having a distal end 204 and a proximal end 206. Distal end 204 is implanted into humerus 60, as shown in FIGS. 17-20, while proximal end 206 remains outside of humerus 60.

Body 202 is generally cylindrical toward distal end 204, where distal end 204 terminates in a tapered tip 208. Distal end 204 includes a plurality of through-openings 210 that allow for the insertion of fixation screws 212 therethrough. Fixation screws 212 extend through humerus 60, into through-openings 210, and back into humerus 60 to secure distal end 204 to humerus 60.

A mid-portion 214 between distal end 204 and proximal end 206 is curved to match the anatomy of humerus 60 and includes a through-opening 220 for securing a capture screw 222. Capture screw 222 can be used to support unstable structures, acting as a gusset support for a proximal fixation screw 224 to reduce any leverage provided by screw 224 due to its length.

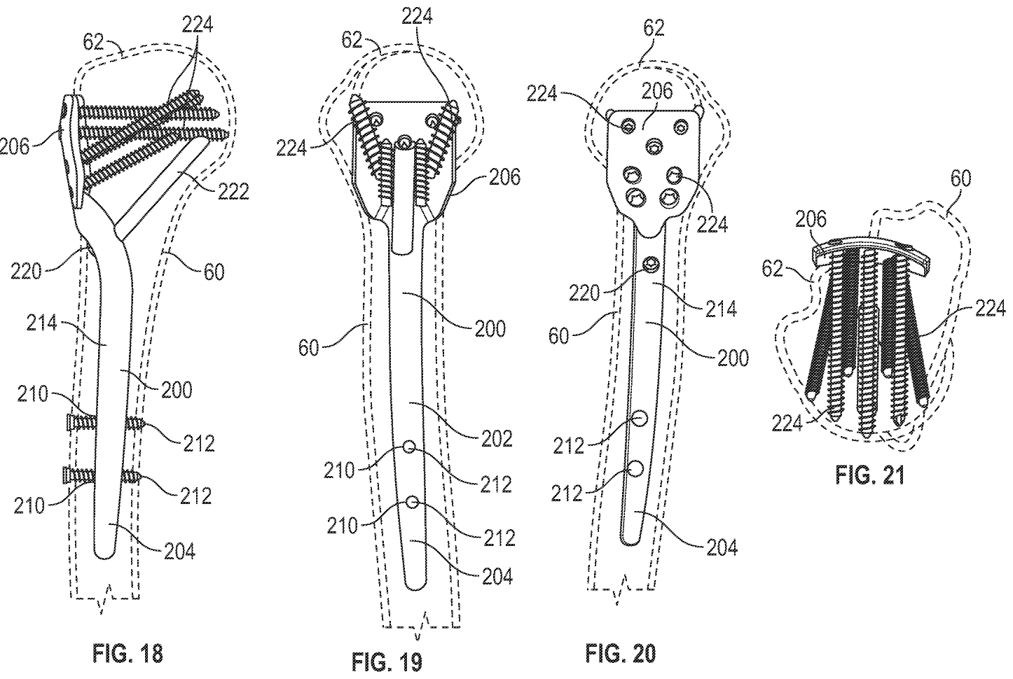

Proximal end 206 is a generally flat plate having a plurality of through-openings 226 formed therethrough. Comparing nail 100 of FIG. 4 with nail 200 of FIG. 16, one can see that nail 200 uses more screws 224 than nail 100 uses screws 124. Additionally, suture holes are provided to capture suture repaired fragments and tendon re-attachment segments Screws 224 are inserted through through-openings 126 and into head/socket 62 of humerus 60, as best seen in FIG. 18.

To use either nail 100 or nail 200, an opening is formed in femur 50 or humerus 60 to allow for insertion of body 102, 202. After nail 100, 200 is inserted, screws 112, 126, 212, 226 and capture screw 122, 222 are inserted though femur 50/humerus 60 and into nail 100, 200 to secure nail 100, 200 to femur 50, humerus 60.

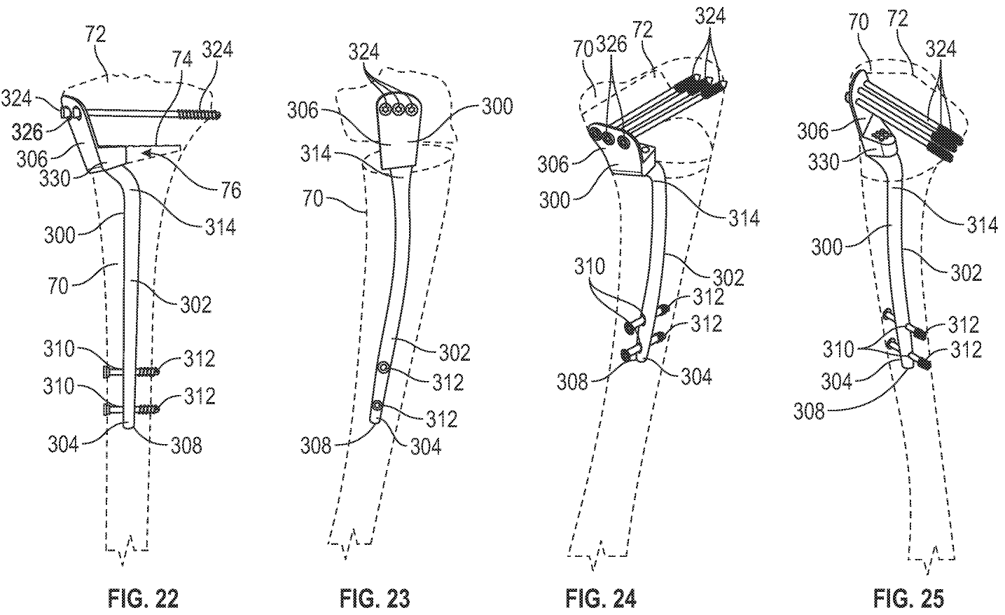

Referring to FIGS. 22-25, an alternative embodiment of an inside-out humerus nail 300 (“nail 300”) is shown. Nail 300 is shown after implantation in tibia 70. Nail 300 is similar to nail 100 and includes an elongate body 302 having a distal end 304 and a proximal end 306. Distal end 304 is implanted into tibia 70, as shown in FIGS. 22, 24, and 25, while proximal end 306 remains outside of tibia 70.

Body 302 is generally cylindrical toward distal end 304, where distal end 304 terminates in a tapered tip 308. Distal end 304 includes a plurality of through-openings 310 that allow for the insertion of fixation screws 312 therethrough. Fixation screws 312 extend through tibia 70, into through-openings 310, and back into tibia 700 to secure distal end 304 to tibia0.

A mid-portion 314 between distal end 304 and proximal end 306 is curved to match the anatomy of tibia 70 and includes a wedge 330 just below proximal end 306. Wedge 330 allows for angulation and the nail portion allows some rotational correction of tibia 70. Wedge 330 is designed to allow either autologous, synthetic or allograft bone graft insertion. Wedge 330 can be hollow to allow injectable bone cement to be inserted after nail 300 is inserted and to reduce the need for prepared bone graft wedges.

Proximal end 306 is a generally flat plate having a plurality of through-openings 326 formed therethrough. Screws 324 are inserted through through-openings 326 and into head 72 of tibia 70, as best seen in FIGS. 22, 24, and 25.

Prior to implanting nail 300 into tibia 70, tibia 70 is cut along line 74, shown in FIG. 22, allowing a space 76 to be opened up between head 72 and the remainder of tibia 70. Wedge 330 is inserted into space 76, along with bone graft material (not shown) to enhance growth of bone material into space 76. Wedge 330 is tapered and can have varying dimensions, depending on the anatomy of the patient and the amount of rotational correction of tibia 70 desired.

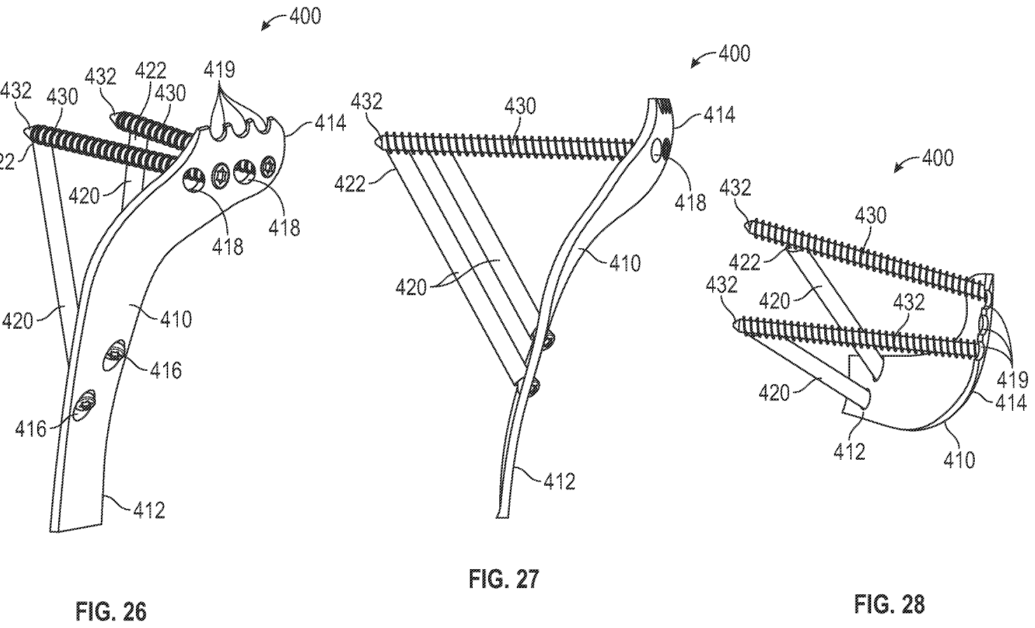

The present invention is a system for repairing an upper tibial fracture, specifically, on the lateral side of the tibia. Referring to FIGS. 26-28, a screw blade capture system 400 (“system 400”) for such a repair according to an exemplary embodiment of the present invention is shown. System 400 is beneficial in supporting repair screws that are used to attach broken upper tibial portions and to support the repair with a plate. The extension length of the screws from the plate, however, result in a cantilevered action on the distal ends of the screws, which, due to the weight of the patient on the upper portion of the tibia, may result in poor healing of the fracture, a snapping of the screws, or some other undesired result. System 400 is a support system to support the distal ends of the screws.

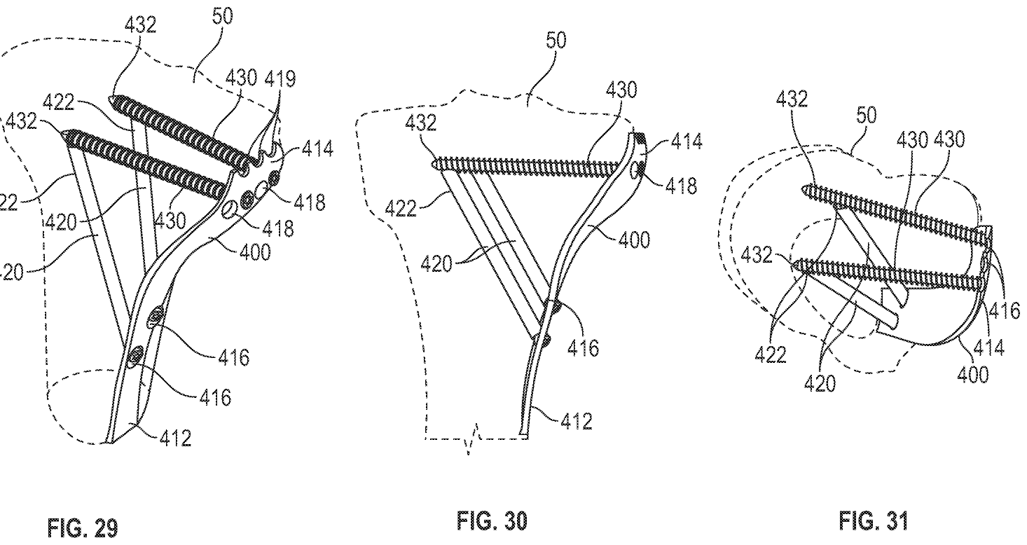

System 400 includes an elongate plate 410 having a lower portion 412 and an upper portion 414. Plate 410 can be contoured to match the contours of an upper tibial portion 70, shown in FIGS. 29-31. Referring back FIGS. 26-28, lower portion 412 includes a plurality of through openings 416 to allow support posts 420 to be inserted therein. Upper portion 414 of plate 410 also includes a plurality of through openings 418, which are used to insert repair screws 430 therethrough. FIG. 29-31 show distal ends 422 of supports 420 each supporting a distal end 432 of a screw 430. Additionally, a plurality of generally U-shaped channels 419 are provided at the top of upper portion 414 of plate 410. Channels 419 can be used to secure additional repair screws (not shown).

Through openings 416, 418 are angled and aligned so that, regardless of which though opening 418 a repair screw 430 is inserted, a support post 420 can be inserted into its through opening 416 and advanced upwardly toward a distal end 432 of screw 430 such that a distal end 422 of support post 420 supports distal end 432 of screw 430, providing a desired cantilever support for screw 430.

It will be further understood that various changes in the details, materials, and arrangements of the parts which have been described and illustrated in order to explain the nature of this invention may be made by those skilled in the art without departing from the scope of the invention as expressed in the following claims.

What is claimed is:

1. A repair plate system comprising:
a body having:
a distal nail portion configured for implantation into a limb bone; and
a proximal plate portion attached to the distal nail portion;
a first plurality of fixation screws configured for insertion into the distal nail portion; and
a second plurality of fixation screws configured for insertion into the proximal plate portion.

2. The repair plate system according to claim 1, wherein the distal nail portion comprises a curved mid portion attached to the proximal plate portion.

3. The repair plate system according to claim 2, further comprising a capture screw configured to act as a gusset support for one of the second plurality of fixation screws.

4. The repair plate system according to claim 3, wherein the capture screw extends through the mid portion.

5. The repair plate system according to claim 1, wherein each of the second plurality of fixation screws are longer than each of the plurality of first fixation screws.

6. The repair plate system according to claim 1, wherein the proximal plate portion comprises a wedge portion attached to the distal nail portion.

7. The repair plate system according to claim 6, wherein the wedge is tapered.

8. The repair plate system according to claim 6, wherein the distal nail portion comprises a curved mid-portion attached to the wedge portion.

9. A repair plate system comprising:
a body having:
a distal nail portion configured for implantation along a limb bone; and
a proximal plate portion attached to the distal nail portion;
a plurality of fixation screws configured for insertion into the proximal plate portion; and
a capture screw configured to act as a gusset support for one of the plurality of fixation screws.

10. The repair plate system according to claim 9, wherein the capture screw extends through the distal nail portion.

11. The repair plate system according to claim 9, wherein the proximal plate portion is contoured to conform to a bone portion.

12. The repair plate system according to claim 9, wherein the proximal plate portion comprises a plurality of generally U-shaped channels.

13. The repair plate assembly according to claim 9, wherein the distal nail portion is configured to extend along an exterior of the limb bone.

14. The repair plate assembly according to claim 9, wherein the distal nail portion is configured to extend along an interior of the limb bone.

15. A repair plate system comprising:
a body having:
a distal nail portion configured for implantation along a limb bone; and
a proximal plate portion attached to the distal nail portion;
a first set of fixation screws configured for insertion into the body;
a second set of fixation screws configured for insertion into the body; and
a capture screw configured to act as a gusset support for one of the sets of fixation screws.

16. The repair plate system according to claim 15, wherein the capture screw is configured to extend through the distal nail portion.

17. The repair plate system according to claim 15, wherein the second set of fixation screws is configured for insertion into the distal nail portion.

18. The repair plate system according to claim 17, wherein the first set of fixation screws is configured for insertion in the proximal plate portion.

19. The repair plate assembly according to claim 15, wherein the second set of fixation screws is configured for insertion into the proximal plate portion.

20. The repair plate assembly according to claim 17, wherein the first set of fixation screws is configured for insertion in the proximal plate portion.
